(19) United States
(12) Patent Application Publication
(10) Pub. No.: US 2026/0357319 A1
(43) Pub. Date: Nov. 12, 2026

(54) Methods for Improving Production of Natural Ketones

(71) Applicants: Agency for Science, Technology and Research, Singapore (SG); National University of Singapore, Singapore (SG); CNRS@CREATE, Singapore (SG); Centre national de la recherche scientifique, Paris (FR); Institut National de Recherche Pour L'Agriculture, L'Alimentation et L'Environnement, Paris (FR); Institut National des Sciences Appliquées de Toulouse (INSA Toulouse), Toulouse (FR)

(72) Inventors: Xixian Chen, Singapore (SG); Deepti Sharma, Singapore (SG); Wen Shan Yew, Singapore (SG); Bo Xue, Singapore (SG); Isabelle Andre, Toulouse (FR); Sophie Bozonnet, Toulouse (FR); Magali Remaud-Simeon, Toulouse (FR); Jérémy Esque, Toulouse (FR)

(21) Appl. No.: 19/130,748

(22) PCT Filed: Nov. 17, 2023
(86) PCT No.: PCT/SG2023/050768

§ 371 Date: May 16, 2025

(30) Foreign Application Priority Data
Nov. 21, 2022 (SG) 10202260151P

(51) Int. Cl. C12Q 1/26; C12N 1/20; C12R 1/01

(52) U.S. Cl. CPC C12Q 1/26; C12N 1/20; C12Y 113/00; C12R 2001/01

(57) ABSTRACT

The invention relates to a cell-based method of identifying one or more enzyme variants that produces an increased level of a ketone. The method of the invention involves incubating cells comprising a vector encoding one or more enzyme variants with para-methox-2-amino benzamidoxime, measuring the signal emitted and comparing the signal to cells comprising a vector encoding a reference enzyme. A system for identifying one or more enzyme variants, and genetically engineered enzyme variants that produce increased levels of a ketone are also disclosed.

### CROSS-REFERENCE TO RELATED APPLICATIONS

This application claims the benefit of priority of Singapore application No. 10202260151P, filed 21 Nov. 2022, the contents of it being hereby incorporated by reference in its entirety for all purposes.

### FIELD OF THE INVENTION

The invention is in the field of biotechnology. The invention relates to improved methods for producing ketones.

### BACKGROUND OF THE INVENTION

Ketones are a class of organic compounds that possess a functional group with the structure R—C(═O)—R′, where R and R′ are carbon-containing substituent groups. Many ketones occur naturally, produced by plants as constituents of essential oils. Several such ketones are in-demand commercially and industrially due to their desirable properties such as fragrance and scent. One such compound, β-ionone, has a woody, violet-like aroma and is the major component of rose ketones. β-ionone belongs to the apocarotenoid family of plant-based secondary metabolites. In nature, it is produced by oxidative cleavage of β-carotene. It has gained commercial interest due to its use as an aromatic agent in the food and fragrance industry. β-ionone and its derivatives have also gained importance in the healthcare industry.

Traditional methods for producing natural ketones involve extraction from natural sources or chemical synthesis. Since ketones such as β-ionone are produced in very minute quantities (less than ng/Kg fresh weight) in nature, other biosynthetic methods have been developed, including lipoxygenase-mediated, peroxidase-mediated and in situ CCD1-mediated oxidative cleavage of carotenoid.

However, current practices to produce natural ketones are not environmentally sustainable and have high production costs. Naturally existing enzymes are not tailored to handle heavy metabolite flux and need optimization to achieve efficient production. Additionally, screening the newly tailored enzymes for enhanced ketone production using the traditional extraction and GC/MS analysis is not feasible in a high-throughput manner necessary for identifying enzyme variants with the desired traits. As such, there is a need to develop a method which ameliorates or overcomes these disadvantages.

### SUMMARY

In one aspect, there is provided a cell-based method of identifying one or more enzyme variants that produces an increased level of a ketone as compared to a reference enzyme comprising the steps of:
(a) culturing one or more cells transformed with an expression vector comprising a polynucleotide sequence encoding one or more enzyme variants;
(b) incubating the one or more cells with para-methoxy-2-amino benzamidoxime (PMA);
(c) measuring the level of fluorescence signal emitted by the one or more cells;
(d) comparing the level of fluorescence signal from step c) with a fluorescence signal measured in a cell transformed with an expression vector comprising a polynucleotide sequence encoding the reference enzyme; and
(e) identifying the one or more enzyme variants when the level of fluorescence signal measured in the cell is increased compared to the reference enzyme.

In another aspect, there is provided a system for identifying one or more enzyme variants that produces an increased level of a ketone as compared to a reference enzyme comprising:
(a) purifying and crystalizing the reference enzyme;
(b) identifying one or more active binding sites and/or one or more sites involved in accessibility to the one or more active binding sites;
(c) introducing one or more amino acid mutations at the active binding sites and/or accessibility sites to generate the one or more enzyme variants;
(d) subjecting the one or more enzyme variants from step c) to the cell-based method described herein to identify the one or more enzyme variants that produces the increased level of ketone.

In yet another aspect, there is provided a genetically engineered enzyme variant identified using the system described herein.

In yet another aspect, there is provided a genetically engineered CCD1 enzyme comprising one or more mutations, wherein the one or more mutations is a substitution at the one or more amino acid positions selected from the group consisting of A191, A400, C354, E408, F303, F530, H241, I171, V179, L356, L423, N339, N377, Q309, Q415, R405, S189, S220, S240, T173, T242, T411, T412, T421, V227, V401 and V429 of a wild type OfCCD1 (SEQ ID NO: 1).

In yet another aspect, there is provided a polypeptide sequence encoding the genetically engineered enzyme as described herein.

In yet another aspect, there is provided a polynucleotide sequence encoding the polypeptide sequence as described herein.

In yet another aspect, there is provided a vector comprising the polynucleotide sequence as described herein.

In yet another aspect, there is provided a host cell comprising the vector as described herein.

### DEFINITIONS

As used herein, a “cell-based method” or a “cell-based assay” refers to a method or assay that is performed on one or more whole cells, or live cells.

As used herein, the term “ketone” refers to a member of the class of organic compounds containing one or more functional groups composed of a carbon atom covalently bonded to an oxygen atom, denoted by the structure R—C(═O)—R′, where R and R′ are carbon-containing substituent groups.

As used herein, the term “isoprenoid” or “terpenoid” are interchangeable terms describing hydrocarbons that contain 1 or multiple isoprene units, as well as their derivatives. Biologically, all isoprenoids are derived from two 5-carbon precursors, namely isoprenyl pyrophosphate and dimethylallyl pyrophosphate. Terpenoids can be further divided into subclasses depending on the number of isoprene units: 1 unit, hemiterpenoid; 2 units, monoterpenoids; 3 units, sesquiterpenoids; 4 units, diterpenoids; 6 units, triterpenoids; 8 units, tetraterpenoids or carotenoids.

As used herein, the term “norisoprenoid” refers to oxidative degradation products of terpenoids. Apocarotenoids are a class of norisoprenoid that are oxidative degradation products of carotenoids. Both apocarotenoids and carotenoids are considered as terpenoids.

As used herein, the term “variant” refers to a modification in the amino acid residues of a polypeptide. The modification in the amino acid residues includes mutation, truncation, translocation, substitution, deletion and insertion, resulting in the alteration of the activity of the polypeptide. The region of the polypeptide that is modified is typically involved in accessibility or binding of ligands in an active site of the polypeptide. The polypeptide may be modified to enhance binding of certain ligands and/or decrease binding of certain ligands.

As used herein, the term “about”, in the context of concentrations of components of the formulations, optical density, time period, typically means+/−10% of the stated value.

### BRIEF DESCRIPTION OF THE DRAWINGS

The invention will be better understood with reference to the detailed description when considered in conjunction with the non-limiting examples and the accompanying drawings, in which:

FIG. 1 shows a schematic diagram of the biosynthetic pathway for β-ionone. FIG. 1A shows that CCD1 can convert lycopene to psi-ionone and/or MHO, and CCD1 can convert carotene to β-ionone, and that carotene is downstream from lycopene in the pathway. FIG. 1B shows the enzymatic products of CCD1 on lycopene and on β-carotene. FIG. 1C shows the reaction mechanism of the PMA assay used to determine the concentration of β-ionone produced by whole-cell biocatalysis from simple carbon source. Abbreviations: CCD1, carotenoid cleavage dioxygenase 1; MHO, 6-methyl-5-hepten-2-one; PMA, para-methoxy-2-amino benzamidoxime.

FIG. 2 shows a 3D structure of OfCCD1 with labels marking the amino acid residues involved in ligand binding, as determined by X-ray diffraction data with a crystal structure resolved to 2.3 Å. The amino acid residues A191 and E408, which are involved in modulating OfCCD1 specificity and activity, are labeled.

FIG. 3 shows the features of PMA fluorescence in cell culture medium. FIG. 3A shows a comparison of PMA fluorescence at λₑₓ=380 nm, λₑₘ=520 nm between water, LB, ZYM, and R-media, before and after incubation with PMA. FIG. 3B shows that the fluorescence of PMA in a cell culture is positively correlated with the cells being transformed with OfCCD1 expression vectors, and is positively correlated with the amount of β-ionone in the culture as determined by gas chromatography. FIG. 3C shows that the PMA fluorescence is correlated to changes in β-ionone production of the transformed cells when one or more amino acids involved in ligand binding in the CCD1 was mutated. Abbreviations: FIG. 3A LB, Luria broth; ZYM, ZYM-5052 media, OfCCD1, Osmanthus fragrans CCD1; FIG. 3B OfCCD1, 6×-His tagged OfCCD1; TrxA-OfCCD1, TrxA tagged OfCCD1; FIG. 3C FN, false negative; FP, false positive; TN, true negative; TP, true positive.

FIG. 4 shows the comparison between wtOfCCD1 and genetically engineered mutants of CCD1 with respect to the production of β-ionone and psi-ionone. FIG. 4A shows the fold change in production of β-ionone and psi-ionone of four specific CCD1 mutants compared to wtOfCCD1. FIG. 4B shows the enzyme catalytic parameters and activity profile of CCD1 mutants compared to wtOfCCD1 when the substrate β-Apo-8′ carotenal is supplied. Abbreviations: wtOfCCD1, wild type Osmanthus fragrans CCD1 Abbreviations: AMEQ, OfCCD1 with A191M/E408Q double mutant; AGEQ, OfCCD1 with A191G/E408Q double mutant.

FIG. 5 Shows a sequence alignment of three CCD1 homologs isolated from plants. FIG. 5A shows the alanine residue (boxed) involved in ligand binding that is conserved between the PhCCD1, OfCCD1, and VvCCD1 homologs. FIG. 5B shows the fold change in β-ionone and psi-ionone production of the mutant enzymes compared to their wild type homologs when the alanine residue is mutated. Abbreviations: PhCCD1, Petunia hybrida CCD1; OfCCD1, Osmanthus fragrans CCD1; VvCCD1, Vitis vinifera CCD1

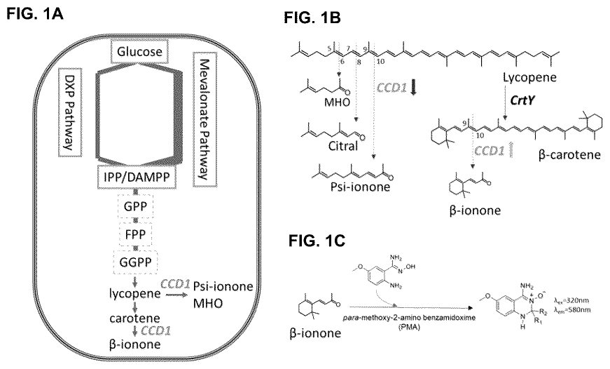

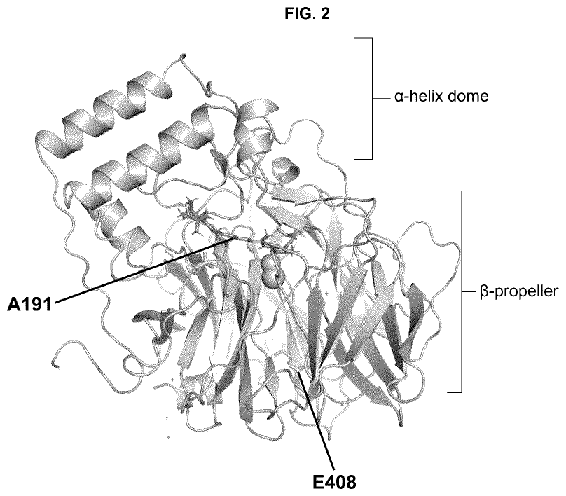

### DETAILED DESCRIPTION OF THE PRESENT INVENTION

In one aspect, the invention relates to a cell-based method of identifying one or more enzyme variants that produces an increased level of a ketone as compared to a reference enzyme comprising the steps of:
(a) culturing one or more cells transformed with an expression vector comprising a polynucleotide sequence encoding one or more enzyme variants;
(b) incubating the one or more cells with para-methoxy-2-amino benzamidoxime (PMA);
(c) measuring the level of fluorescence signal emitted by the one or more cells;
(d) comparing the level of fluorescence signal from step c) with a fluorescence signal measured in a cell transformed with an expression vector comprising a polynucleotide sequence encoding the reference enzyme; and
(e) identifying the one or more enzyme variants when the level of fluorescence signal measured in the cell is increased compared to the reference enzyme.

The one or more cells of the present invention may be a cell that is a suitable host for expression vectors. For example, the cell may be a plant cell, a prokaryotic cell or a eukaryotic cell.

The one or more cells of the cell-based method or assay may be cultured in a culture medium. It would generally be understood that the culture medium may be supplemented with one or more components that are suitable for maintenance of the cells, growth of cells, or for performing the cell-based method of the invention, or a combination thereof.

In one example, the cells may be cultured in a culture medium including but not limited to water, Luria Broth, R-media, and auto-induction media. Auto-induction media may contain one or more inducers suitable for inducing an inducible expression vector. In one example, the auto-induction media is ZYM-5052 (or ZYM) for auto induction.

In one example, the culture medium may be R-media.

The one or more cells cultured in the culture medium may be transformed with an expression vector comprising a comprising a polynucleotide sequence encoding one or more enzyme variants. In one example, the polynucleotide sequence encoding the one or more enzyme variants is operably linked to a promoter. The promoter may be an inducible or constitutive promoter.

In one example, the polynucleotide sequence encoding the one or more enzyme variants is operably linked to an inducible promoter.

In one example, the polynucleotide sequence encoding the reference enzyme is operably linked to a promoter. The promoter may be an inducible or constitutive promoter. In one example, the polynucleotide sequence encoding the reference enzyme is operably linked to an inducible promoter.

The culture medium in which the one or more cells are cultured in may be supplemented with an inducer. In some examples, the inducer may be supplemented at the beginning of the culture process. For example, the inducer may be supplemented in the medium prior to just after inoculation of the medium with the one or more cells. In another example, the inducer may be supplemented in the medium when the cells are at a predetermined cell density or confluency.

In an example, the medium is supplemented with the inducer when the cells are at an Optical Density at 600 nm (OD600) of about 0.05 to 1. For example, the OD600 may be about 0.05, 0.06, 0.07, 0.08, 0.09, 0.10, 0.2, 0.3, 0.4, 0.5, 0.6, 0.7, 0.8, 0.9 or 1.0.

It would be generally understood that the cell culture medium may be supplemented by any inducer that is able to induce the inducible promoter in the transformed cell. For example, the inducer may be isopropyl β-D-1-thiogalactopyranoside (IPTG) or lactose. In one example, the inducer is lactose.

The total concentration of the inducer in the cell culture may be adjusted to a desired concentration to induce the promoter. In some examples, the concentration of lactose is between about 10-50 mM. For example, the concentration of lactose may be about 10 mM, 15 mM, 20 mM, 25 mM, 30 mM, 35 mM, 40 mM, 45 mM, or 50 mM. In one example, the concentration of lactose is about 30 mM.

In some examples, the concentration of IPTG is between about 0.01-1.0 mM. For example, the concentration of IPTG may be about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9 or 1.0 mM. In one example, the concentration of IPTG is about 0.1 mM.

The production of ketone by the transformed cells of the method as described herein can be detected by a reagent, such as para-methoxy-2-amino benzamidoxime (PMA). In an example, the reagent may emit a detectable signal in the presence of a ketone. For example, the detectable signal may be a fluorescent signal, a chemiluminescent signal, a colorimetric signal, or combinations thereof. The level of the detectable signal may be indicative of the amount of ketone produced by the enzyme variant.

In the cell-based method of the invention, the one or more cells transformed with an expression vector is incubated with para-methoxy-2-amino benzamidoxime (PMA).

In an example, the cells are incubated with PMA at a concentration of between about 5 mM-15 mM. For example, the concentration of the PMA may be about 5 mM, 6 mM, 7 mM, 8 mM, 9 mM, 10 mM, 11 mM, 12 mM, 13 mM, 14 mM, or 15 mM. In one example, the concentration of PMA is about 10 mM.

The cells may be incubated with PMA for a predetermined period of time. In an example, the cells are incubated with the PMA for a duration of about 5 minutes to about 3 hours. For example, the cells may be incubated with PMA for a duration of about 5 minutes, 10 minutes, 20 minutes, 30 minutes, 40 minutes, 50 minutes, 60 minutes, 90 minutes, 120 minutes, 150 minutes, or 180 minutes. In one example, the cells are incubated with PMA for about 10 minutes.

The method of the invention may be used to measure the quantity of a ketone produced by the transformed cells. The ketone may be a compound naturally produced by a plant. For example, the ketone may be selected from the group of compounds consisting of a carotenoid, an apocarotenoid, a terpenoid, a triterpenoid and a norisoprenoid. In one example, the ketone may be an apocarotenoid.

In some examples, the norisoprenoid is selected from the group consisting of β-ionone, 6-methyl-5-hepten-2-one (MHO), irone, α-ionone and psi-ionone. In other examples, the apocarotenoid is selected from the group consisting of β-ionone, 6-methyl-5-hepten-2-one (MHO), α-ionone and psi-ionone. In one example, the apocarotenoid is β-ionone.

In the method of the invention, the level of fluorescence emitted by the one or more cells transformed with an expression vector comprising a polynucleotide sequence encoding one or more enzyme variants may be measured and compared with the level of fluorescence emitted by one or more cells transformed with an expression vector comprising a polynucleotide sequence encoding a reference enzyme.

The level of fluorescence emitted by the cells transformed with an expression vector comprising a polynucleotide sequence encoding one or more enzyme variants may in turn be indicative of the ketone production yield of the enzyme variant encoded by the vector. The ketone production yield of the one or more enzyme variants that produce a ketone may be compared with the ketone production yield of a reference enzyme.

As such, the method disclosed herein may identify enzyme variants that produce an increased and/or decreased yield of one or more ketones compared to a reference enzyme.

In one example, the enzyme variant identified may produce an increased β-ionone yield as compared to the reference enzyme. In another example, the enzyme variant identified may produce a decreased psi-ionone yield as compared to the reference enzyme. In yet another example, the enzyme variant may produce an increased β-ionone yield and a decreased psi-ionone yield compared to the reference enzyme. In some examples, the enzyme variant identified may produce an increased β-ionone yield of at least 20 percent compared to the reference enzyme. In some examples, the enzyme variant identified may produce an increased β-ionone yield of between about 1- to 15-fold as compared to the reference enzyme. In other examples, the enzyme variant identified may produce a decreased psi-ionone yield of a reduced psi-ionone yield of at least 10 percent compared to the reference enzyme. In yet other examples, the enzyme variant identified may produce a decreased psi-ionone yield of between about 1- to 5-fold as compared to the reference enzyme. In one example, the β-ionone yield is increased by about 1.4-fold as compared to the reference enzyme and the psi-ionone yield is reduced by about 2-fold as compared to the reference enzyme. In another specific example, the β-ionone yield is increased by at least 40 percent and the psi-ionone yield is reduced by at least 30 percent as compared to the reference enzyme.

In one example, the identified enzyme variant may be further validated for ketone production using gas chromatography mass spectrometry (GCMS).

The one or more cells used in the cell-based method described herein may be a bacterial cell.

In one example, the cell may be an Escherichia coli cell. In some examples, the Escherichia coli cell may be an industrial strain. In one example, the E. coli strain may include but is not limited to the BL21 DE3 strain, the MG1655DE3 strain, the DH1 strain, the MG1655 strain, the DH5A strain, the K12 strain, the W3110 strain, or combinations thereof.

The cell-based method may be performed on a range of sample quantities or enzyme variants (throughputs). For example, the cell-based method of the invention may be performed on a single sample or enzyme variant, or multiple samples or enzyme variants. In some examples, the method may be performed on samples or enzyme variants contained in a test tube, a cuvette, a petri dish, or a multi-well test plate, or in a multitude of such vessels. In some examples, 1 to 10s of samples or enzyme variants may be screened (low throughout). In other examples, 100s to 1000s of samples or variants may be screened (medium throughput). In other examples, between 1×10³-1×10⁸ samples or variants may be screened (high throughput).

The cell-based method may be performed using various vessels. In an example, the cell-based method may be performed on one or more sample or wells of a multi-well test plate. For example, the method may be performed on a 6-well plate, a 12-well plate, a 24-well plate, a 48-well plate, a 96-well plate, a 192-well plate, a 384-well plate, a 1536-well plate, a 3456-well plate or a 6144-well plate. In one example, the method is performed on a 96-well test plate.

The cell-based method may be performed on a single plate or on multiple plates, in a single batch or in multiple batches.

The one or more enzyme variants identified with the cell-based method of the present invention may be generated by mutation of a reference enzyme. In an example, the mutation may be an amino acid mutation. The amino acid mutation may be at one or more active binding sites, and/or one or more sites involved in accessibility to the one or more active binding sites.

The reference enzyme may be any enzyme to which the one or more enzyme variants may be compared with. For example, the reference enzyme may be a wild type enzyme that produces the ketone, or an enzyme with a known production yield of the ketone. In one example, the enzyme with a known production yield of the ketone may be encoded by a polynucleotide sequence that has been codon optimized. It would be generally understood that a codon optimized polynucleotide sequence may result in increased expression of the polypeptide encoded by the optimized polynucleotide.

In one example, the enzyme variant may be a CCD1 variant.

The cell-based method may be conducted in vivo. For example, the method may be performed on one or more whole cells, or live cells.

In another aspect, provided herein is a cell-based method of identifying one or more enzyme variants that produces an increased level of β-ionone as compared to a reference enzyme comprising the steps of:
(a) culturing one or more cells transformed with an expression vector comprising a polynucleotide sequence encoding one or more enzyme variants;
(b) incubating the one or more cells with para-methoxy-2-amino benzamidoxime (PMA);
(c) measuring the level of fluorescence signal emitted by the one or more cells;
(d) comparing the level of fluorescence signal from step c) with a fluorescence signal measured in a cell transformed with an expression vector comprising a polynucleotide sequence encoding the reference enzyme; and
(e) identifying the one or more enzyme variants when the level of fluorescence signal measured in the cell is increased compared to the reference enzyme, wherein the enzyme variant is a CCD1 variant and wherein the reference enzyme is a wild type CCD1 enzyme.

In another aspect, the invention relates to a system for identifying one or more enzyme variants that produces an increased level of a ketone as compared to a reference enzyme comprising the steps of:
(a) purifying and crystalizing the reference enzyme;
(b) identifying one or more active binding sites and/or one or more sites involved in accessibility to the one or more active binding sites;
(c) introducing one or more amino acid mutations at the active binding sites and/or accessibility sites to generate the one or more enzyme variants; and
(d) subjecting the one or more enzyme variants from step c) to the cell-based method as described herein to identify the one or more enzyme variants that produces the increased level of ketone.

The system provided herein can be used to identify enzyme variants that have an increased binding affinity for a substrate and/or increased catalytic activity compared to a reference enzyme.

For example, one or more active binding and/or one or more sites involved in accessibility to the one or more active binding sites may be identified using an in silico method. In one example, the method may be a structure-guided in silico molecular modelling method. For example, the in silico method may include but is not limited to PyMol 2.5, Modeller 10.0 or YASARA 21.12.19.

In another example, the one or more active binding and/or one or more sites involved in accessibility to the one or more active binding sites may be mutated to generate an enzyme variant. In one example, the mutation may be a substitution, insertion, or deletion of an amino acid.

In another aspect, provided herein is a genetically engineered enzyme variant identified using the system as described herein.

In an example, the genetically engineered enzyme variant is an apocarotenoid pathway enzyme. In a further example, the apocarotenoid enzyme is carotenoid cleavage dioxygenase (CCD).

In some examples, the CCD may include but is not limited to CCD1, CCD2, CCD4, CCD7 and CCD8.

In one example, the CCD enzyme may be CCD1 or CCD4. In one example, the CCD is CCD1.

The genetically engineered enzyme variant CCD1 may in some examples be mutated at one or more amino acid positions.

The CCD gene may be a plant CCD gene. In an example, the CCD1 gene may be a plant CCD1 gene that includes but is not limited to Petunia hybrida (PhCCD1), Vitis vinifera (VvCCD1) and Osmanthus fragrans (OfCCD1).

The genetically engineered CCD1 may comprise one or more mutations in its amino acid sequence. In one example, the one or more mutations is a substitution at one or more amino acid positions selected from the group consisting of A191, A400, C354, E408, F303, F530, H241, I171, V179, L356, L423, N339, N377, Q309, Q415, R405, S189, S220, S240, T173, T242, T411, T412, T421, V227, V401, V429 and combinations thereof of a wild type OfCCD1 (SEQ ID NO: 1). In another example, the one or more mutations is a substitution at one or more amino acids equivalent to the positions selected from the group consisting of A191, A400, C354, E408, F303, F530, H241, I171, V179, L356, L423, N339, N377, Q309, Q415, R405, S189, S220, S240, T173, T242, T411, T412, T421, V227, V401, V429 and combinations thereof of a wild type OfCCD1 (SEQ ID NO: 1). It is generally understood that the term “equivalent”, when used in reference to the position of an amino acid in an amino acid sequence, refers to a position of the amino acid in the sequence of a given amino acid sequence, which corresponds in position (in either primary or tertiary structure) to a position in a reference amino acid sequence. Such equivalent positions in a particular sequence can be determined using methods known in the art, for example based on sequence alignment against the reference sequence or by comparing experimentally revealed or predicted 3D-structures of corresponding proteins. For example, amino acid position 191 of the OfCCD1 polypeptide as set forth in SEQ ID NO: 1 would be understood to be equivalent to amino acid position 194 of the PhCCD1 polypeptide as set forth in SEQ ID NO: 2.

In one example, the mutation in CCD1 may be A191 G, A191 L, A191M, A400V, C354S, E408P, E408Q, F303Y, I171T, L3561, L356M and R405K, or combinations thereof.

In one example, the genetically engineered CCD1 variant comprising the one or more mutations may produce an increased β-ionone yield. In another example, the genetically engineered CCD1 variant comprising the one or more mutations may produce a decreased psi-ionone yield compared to a wild type CCD1. In yet another example, the genetically engineered CCD1 variant comprising the one or more mutations may produce an increased β-ionone yield and decreased psi-ionone yield compared to a wild type CCD1.

In one example, the genetically engineered enzyme variant that produces an increased level of a ketone may have an increased binding affinity for a substrate and/or increased catalytic efficiency compared to a reference enzyme.

In some examples, the substrate is selected from the group comprising β-apo-8′ carotenal, β-apo-10′ carotenal, 8′-apo-lycopenal, 10′-apo-lycopenal, β-carotene and lycopene. In one example, the substrate is β-apo-8′ carotenal.

In one example, the genetically engineered CCD1 enzyme comprising the mutations at amino acid positions A191 and E408 has increased binding specificity for β-apo-8′ carotenal or increased catalytic efficiency or increased β-ionone production or combinations thereof compared to the wild type CCD1.

In an example, the genetically engineered CCD1 enzyme comprises one or more mutations, wherein the one or more mutations is a substitution at the one or more amino acid positions selected from the group consisting of A191, A400, C354, E408, F303, F530, H241, I171, V179, L356, L423, N339, N377, Q309, Q415, R405, S189, S220, S240, T173, T242, T411, T412, T421, V227, V401 and V429 of a wild type OfCCD1 (SEQ ID NO: 1).

In another aspect, provided herein is a polypeptide sequence encoding the genetically engineered CCD1 enzyme as described herein.

In another aspect, provided herein is a polynucleotide sequence encoding the polypeptide sequence as described herein.

In another aspect, provided herein is a vector comprising the polynucleotide sequence as described herein.

In another aspect, provided herein is a host cell comprising the vector as described herein.

In some examples the host cell may be a bacterial cell. In some examples, the bacterial cell may be an Escherichia coli cell. For example, the Escherichia coli cell may be selected from the group consisting of a BL21 DE3 strain, a MG1655DE3 strain and any industrial strain (such as DH1, MG1655, DH5A, K12 and W3110).

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

Non-limiting examples of the invention and comparative examples will be further described in greater detail by reference to specific Examples, which should not be construed as in any way limiting the scope of the invention.

### Example 1: OfCCD1 Structure and Modelling of Complexes

OfCCD1 was cloned, expressed and purified from a bacterial system. Briefly, codon optimized gene fragment coding OfCCD1 protein was cloned into pSY5 bacterial expression vector. The vector was used to transform BL21 DE3 E. coli cells. The transformed cells were induced using 0.1 mM isopropyl β-D-1-thiogalactopyranoside (IPTG) followed by incubation at 28 °C overnight. The cells were harvested, lysed and centrifuged. The lysate containing His-tagged OfCCD1 protein was subjected to Ni-NTA affinity column chromatography followed by removal of his-tag and further purification using size exclusion chromatography. Commercially available crystallization kits were used to screen for initial hits. The initial hit condition was further optimized to obtain single large crystals. X-ray diffraction data collection followed by analysis led to a 2.3 Å crystal structure of OfCCD1. The overall 3D structure remains conserved, with 7-blade β-barrel, α-helix dome and active ion centre (FIG. 2).

The OfCCD1 crystal structure shows a Triton X-100 molecule in the substrate tunnel. The Triton X-100 molecule was used as reference for ligand (substrate or products) molecular docking and Nickel (Ni) atom in the ion centre was replaced with iron (Fe) atom. The water molecule at the catalytic site was replaced with two oxygen atoms.

The different 3D models of OfCCD1 in free form or in complex with lycopene (substrate), β-carotene, β-ionone, psi-ionone were then subjected to energy minimization before subjecting them to MD simulations (20-100 ns). Analysis of amino acid residues involved in accessibility or binding of ligands in active site was performed and enabled to identify key amino acid positions to target by mutagenesis to enhance binding of lycopene and β-ionone and decrease binding of psi-ionone.

### Example 2: Development of a High-Throughput Assay for Preliminary Enzyme Screening

LB, ZYM and R-media were tested for minimum background fluorescence with PMA. R-media (4.2 g/L potassium dihydrogen monobasic, 11.24 g/L potassium phosphate dibasic, 1.86 g/L citric acid, 1% v/v of 10M sodium hydroxide, 2 g/L ammonium sulphate, 10 ml/L trace element solution, 0.06 g/L iron (Ill) citrate, 0.5 g/L magnesium sulphate, 0.045 g/L thiamine, 2 g/L glucose, 8 g/L glycerol and 30 mM lactose; the trace element solution (100×) contained 0.25 g/L CoCl₂·6H₂O, 1.5 g/L MnSO₄·4H₂O, 0.15 g/L CuSO₄·2H₂O, 0.3 g/L H₃BO₃, 0.25 g/L Na₂MoO₄·2H₂O, 0.8 g/L Zn(CH₃COO)₂, and 0.84 g/L EDTA, pH 8.0) was chosen for further analysis as it showed the least non-specific fluorescence with and without PMA.

The reaction conditions were optimized using cell cultures with known relative β-ionone production levels. Briefly, BL21 DE3 E. coli cells harboring plasmids for β-carotene production were transformed with vectors expressing either 6×-His tagged OfCCD1, Trx-A tagged OfCCD1 or empty vector, where Trx-A-OfCCD1 containing cells should have highest β-ionone followed by His-OfCCD1 and no β-ionone production in cells without OfCCD1. The cells were cultured overnight in LB media at 37 °C and used for inoculating 1 ml R-media to achieve 0.1 OD600. The cultures were grown for 2 days at 28 °C. 100 μl of culture, cells only or media only was incubated with 1 μl of 1M PMA for 10 min, 1 hour or 2 hours followed by fluorescence measurement (λₑₓ=380 nm/λₑₘ=520 nm). For GC analysis, 100 μl of cell culture was extracted with 100 μl of hexane and peaks corresponding to β-ionone on the chromatograms were normalized with respect to His-OfCCD1 and compared to the PMA assay fluorescence results. Time point analysis showed that 10 minutes of incubation of cell cultures with PMA showed discernible fluorescence readings with respect to ionone (ketone) produced and the results were in agreement with GC analysis.

To further adapt the PMA assay for automation, a few variations were made to the procedure mentioned above. Briefly, competent cells containing plasmid system to produce β-carotene were transformed with OfCCD1 mutant's expression vector. The transformed cells were grown in 1 ml LB overnight shaking at 37 °C in sterile 96-deep well plate and this pre-culture (5% v/v) was used to inoculate 1 ml R-media in sterile 96-deep well plate. Lactose (30 mM) was used for induction and cultures were grown at 28 °C for 2 days. Cells (100 μl) were aliquoted in a 96-well plate (Greiner, Austria) followed by addition of 10 μl of 0.1 M PMA. The plate was incubated for 10 min and emission at 520 nm was measured after excitation at 380 nm. The assay was performed in triplicates using the Biomek i7 robotics system (Beckman coulter, Brea, CA, USA).

### Example 3: In Silico Designing and HTS Assay to Identify Functionally Enhanced Mutants

Using structure-based in silico analysis in combination with stability predictions we identified 27 sites (1171, T173, V179, S189, A191, S220, V227, S240, H241, T242, F303, Q309, N339, C354, L356, N377, A400, V401, R405, E408, T411, T412, T421, Q415, L423, V429 and F530) with a total of 70 corresponding mutants to be of interest for specifically enhancing β-ionone production (Table 1, left column). Dark greys denote a decrease in the PMA signal compared to the reference enzyme, light greys denote an increase in the PMA signal compared to the reference enzyme). 19 mutants (A191 F, A191 G, A191M, A191S, A400V, C354S, E408D, E408P, E408Q, F303L, F303V, F303Y, 1171F, I171T, L3561, L356M, R405K, T421G and V401 S) with over 10% increment in PMA fluorescence were selected and analyzed further using GC/MS (Table 2, darker greys denote a greater deviance from the reference enzyme).

TABLE 1
PMA fluorescence of enzyme variants compared to OfCCD1

| | PMA based | | PMA based | | PMA based |
|---|---|---|---|---|---|
| Mutation | normalization | Mutation | normalization | Mutation | normalization |
| predicted | with respect | predicted | with respect | predicted | with respect |
| by | to wtOfCCD1 | by | to wtOfCCD1 | by | to wtOfCCD1 |
| A191F | 1.13957 | L356I | 1.106544 | S240C | 1.064489 |
| A191G | 1.150764 | L356M | 1.103399 | S240D | 1.046397 |
| A191M | 1.153226 | L356Q | 1.036099 | T173F | 0.967334 |
| A191S | 1.161972 | L423A | 1.046402 | T173L | 1.042626 |
| A400V | 1.146657 | L423G | 1.014441 | T173V | 1.072587 |
| C354P | 1.032555 | N339A | 1.063367 | T242K | 0.984608 |
| C354S | 1.153963 | N339L | 1.066488 | T411F | 1.036935 |
| E408D | 1.10419 | N339T | 1.031684 | T411L | 1.040178 |
| E408P | 1.101002 | N377A | 1.051125 | T411S | 1.08884 |
| E408Q | 1.140937 | N377S | 1.036825 | T411V | 1.062541 |
| E408R | 1.085899 | Q309K | 1.088459 | T412G | 1.045208 |
| F303I | 1.092383 | Q309P | 1.081375 | T412W | 0.998568 |
| F303L | 1.136016 | Q415D | 1.051559 | T421A | 1.064167 |
| F303V | 1.1373 | Q415H | 0.967299 | T421G | 1.164003 |
| F303Y | 1.134332 | Q415N | 1.070834 | V227F | 0.994699 |
| F530L | 1.089808 | Q415S | 1.044423 | V227I | 1.038319 |
| F530T | 1.067087 | Q415T | 1.084183 | V227L | 1.050459 |
| H241A | 1.090635 | R405K | 1.132191 | V227S | 0.98923 |
| H241T | 1.087965 | R405M | 1.078881 | V401L | 1.026496 |
| H241V | 1.062297 | S189D | 1.037579 | V401S | 1.14011 |
| I171F | 1.135499 | S189G | 0.998367 | V429F | 1.047187 |
| I171L | 1.005103 | S189M | 1.0474 | V429I | 0.939085 |
| I171T | 1.104772 | S189T | 0.991214 | wtOfCCD1 | 1 |
| V179I | 0.986734 | S220G | 0.949339 | | |

TABLE 2
Comparison of β-ionone and psi-ionone of enzyme variants

| | | | | | Unexpected |
|---|---|---|---|---|---|
| | | | | | (as β-ionone |
| | | | Expected β- | Expected | increases but |
| | β-ionone | psi-ionone | ionone | psi-ionone | psi-ionone |
| | (normalized) | (normalized) | enhancement | enhancement | decreases) |
| A191F | 0.66 | 0.47 | yes | yes | |
| A191G | 1.18 | 0.4 | yes | yes | + |
| A191M | 1.37 | 1.79 | yes | yes | |
| A191S | 0.83 | 0.75 | yes | yes | |
| A400V | 1.19 | 0.85 | yes | yes | + |
| C354S | 1.21 | 0.48 | yes | yes | + |
| E408D | 1.25 | 1.09 | yes | yes | |
| E408P | 1.23 | 0.47 | yes | yes | + |
| E408Q | 1.31 | 0.62 | yes | yes | + |
| F303L | 0.79 | 0.85 | yes | yes | |
| F303V | 1.26 | 1.44 | yes | yes | |
| F303Y | 1.24 | 0.6 | yes | yes | + |
| I171F | 0.99 | 1.71 | yes | yes | |
| I171T | 1 | 0.65 | yes | yes | + |
| L356I | 1.38 | 0.9 | yes | yes | + |
| L356M | 1.28 | 0.53 | yes | yes | + |
| R405K | 1.13 | 0.99 | yes | yes | + |
| T421G | 0.86 | 0.3 | yes | yes | |
| V401S | 0.05 | 0.75 | yes | yes | |
| wt OfCCD1 | 1 | 1 | baseline β- | baseline psi- | |
| | | | ionone | ionone | |

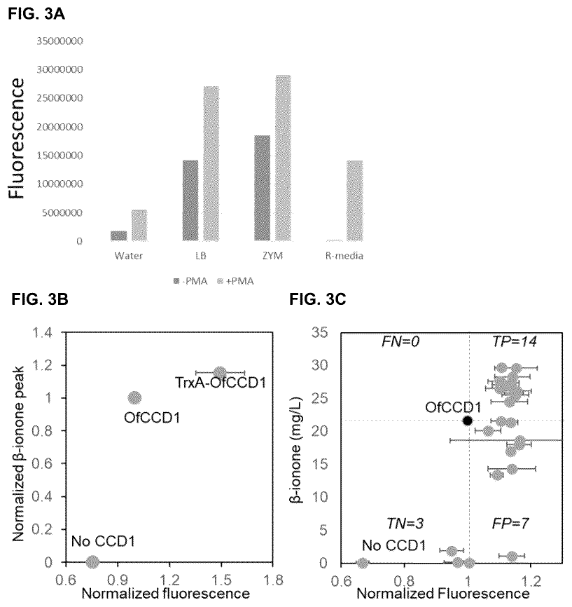

Bacterial cultures with selected OfCCD1 variants were grown in R-media under dodecane layer and expression was induced using 30 mM lactose. After 2-4 days of production the dodecane layer was diluted in hexane (1:20) and the samples were subjected to analysis using Agilent Intuvo 9000 GC system with a DB-WAX Ultra Inert Intuvo GC column module (30 m, 0.25 mm, 0.25 μm) and an Agilent 5977B mass spectrometer. Sample (10:1 split, 250 °C) was injected and developed on column at 80 °C to 200 °C at a ramp rate of 40 °C/min and after a hold of 3 min at 200 °C the ramp rate was increased to 80 °C/min to reach 230 °C followed by a hold for 2 min at 230 °C. Commercially available β-ionone and psi-ionone (Sigma, MI, USA) were used to prepare standard curves. MS Quant analysis software was used to deduce β-ionone and psi-ionone concentrations in the samples. We confirmed that 14 mutants out of 19 had increased β-ionone production (FIG. 3C). These 14 mutants with mutations across 7 sites (A191, L356, E408, F303, C354, A400 and 1171) are currently being investigated for further improvement.

### Example 4: Functionally Enhanced Mutants

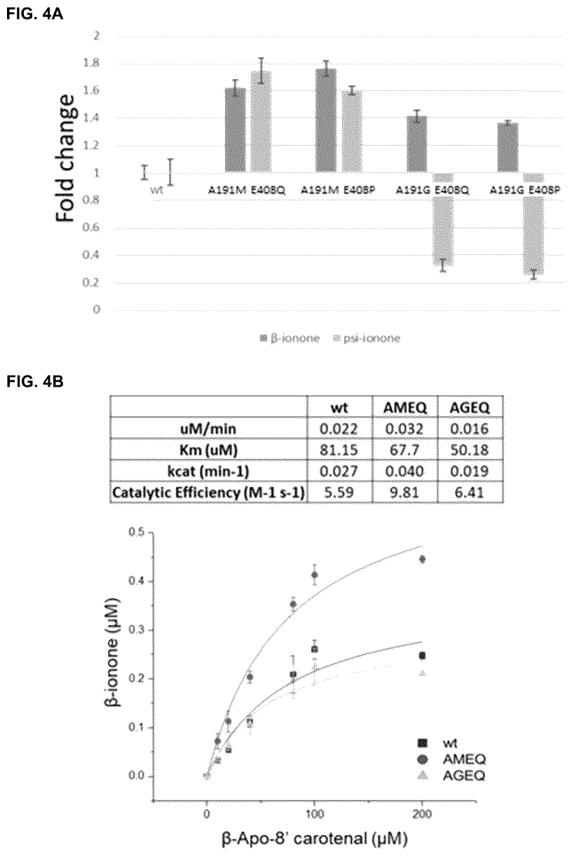

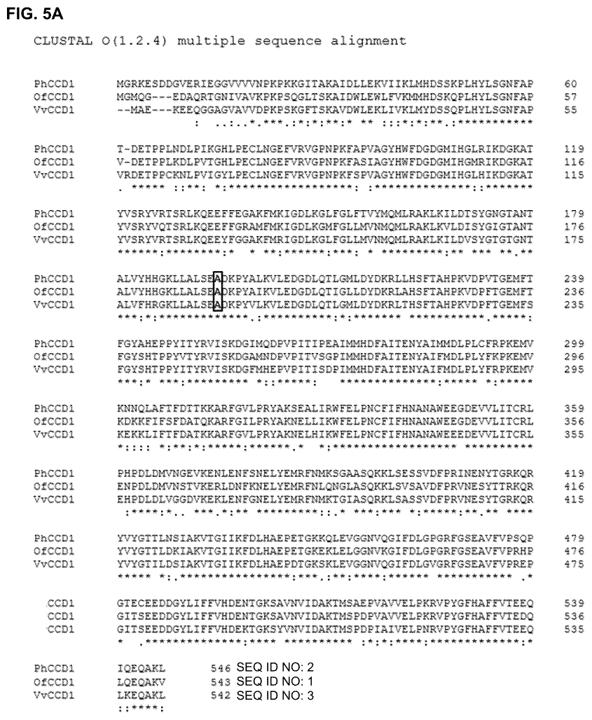

OfCCD1 A191-E408 double mutants have enhanced activity and specificity (FIG. 4A). These double mutants were expressed, purified and crystallized same as wild type OfCCD1. Using β-apo-8′ carotenal as substrate, the enzyme kinetic parameters for wildtype and mutants (best double mutants) were determined. Varying concentrations of β-apo-8′ carotenal were incubated at 37 °C with 5 μg of OfCCD1 variant for 20 min in 100 μl reaction volume (20 mM Tris pH 7.5, 150 mM NaCl, 0.1% TritonX-100). The sample was extracted with 100 μl ethyl acetate (EA) and β-ionone was quantitated as explained above using GC/MS. The results show that the mutants have a lower Km compared to wildtype (FIG. 4B), indicating improved in vivo specificity for the desired substrate.

### Example 5: Regulation of CCD1 Homologs Enzyme Activity

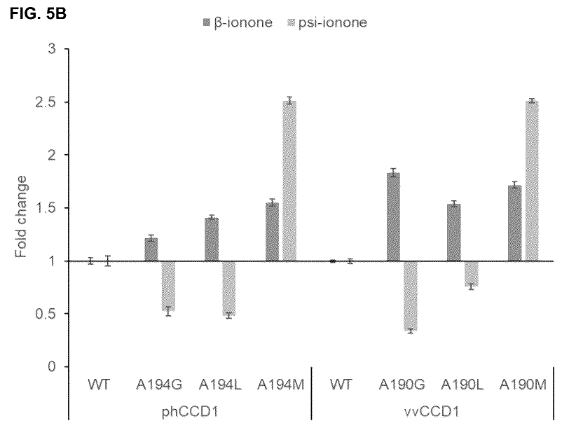

In order to confirm that the sites involved in modulating OfCCD1 activity could similarly regulate the function of other CCD1 homologs, tests were conducted on corresponding Petunia hybrida CCD1 (PhCCD1) and Vitis vinifera CCD1 (VvCCD1) variants (FIG. 5A). OfCCD1 A191G/L/M homologs PhCCD1 A193G/L/M and VvCCD1 A193G/L/M were cloned, expressed and tested for β-ionone and psi-ionone production as described earlier. The results confirmed that PhCCD1 and VvCCD1 variants homologous to A191 G/L homolog mutants have specifically enhanced activity for β-ionone production (FIG. 5B). These results suggest that the sites targeted for mutation in the OfCCD1 can translate well to other CCD1 homologs in regulating enzyme activity and specificity.

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1. A cell-based method of identifying one or more enzyme variants that produces an increased level of a ketone as compared to a reference enzyme comprising the steps of:
(a) culturing one or more cells transformed with an expression vector comprising a polynucleotide sequence encoding one or more enzyme variants;
(b) incubating the one or more cells with para-methoxy-2-amino benzamidoxime (PMA);
(c) measuring the level of fluorescence signal emitted by the one or more cells;
(d) comparing the level of fluorescence signal from step c) with a fluorescence signal measured in a cell transformed with an expression vector comprising a polynucleotide sequence encoding the reference enzyme; and
(e) identifying the one or more enzyme variants when the level of fluorescence signal measured in the cell is increased compared to the reference enzyme.

2. The cell-based method according to claim 1, wherein the culture medium is R-media, optionally wherein the culture medium is supplemented with an inducer, optionally wherein the inducer is supplemented at the beginning of the culture process or the inducer is supplemented to the culture medium when the cells are at a predetermined cell density (OD600), optionally wherein the cells are at an OD600 of between about 0.05 to 1, optionally wherein the inducer is isopropyl β-D-1-thiogalactopyranoside (IPTG) or lactose.

3. The cell-based method according to claim 1 wherein the polynucleotide sequence encoding the one or more enzyme variants and the polynucleotide sequence encoding the reference enzyme are operably linked to an inducible promoter.

4.-5. (canceled)

6. The cell-based method according to claim 2, wherein the total concentration of lactose is between about 10 mM-50 mM, optionally wherein the total concentration of lactose is about 30 mM.

7. The cell-based method according to claim 1, wherein the concentration of PMA is between about 5 mM-15 mM, optionally wherein the concentration of PMA is about 10 mM, optionally wherein the PMA is incubated with the cells for a duration of about 5 minutes to about 3 hours, optionally wherein the PMA is incubated with the cells for about 10 minutes.

8.-9. (canceled)

10. The cell-based method according claim 1, wherein the ketone is selected from the group consisting of a carotenoid, an apocarotenoid, a terpenoid, a triterpenoid and a norisoprenoid, optionally wherein the ketone is an apocarotenoid.

11. The cell-based method according to claim 10, wherein the norisoprenoid is selected from the group consisting of β-ionone, 6-methyl-5-hepten-2-one (MHO), irone, α-ionone and psi-ionone, optionally wherein the apocarotenoid is selected from the group consisting of β-ionone, 6-methyl-5-hepten-2-one (MHO), α-ionone and psi-ionone, optionally wherein the apocarotenoid is β-ionone.

12. The cell-based method according to claim 1, wherein the one or more cells is a bacterial cell, optionally wherein the bacterial cell is an Escherichia coli cell.

13. (canceled)

14. The cell-based method according to claim 1, wherein the one or more enzyme variants is generated by mutation of the reference enzyme, optionally when the mutation is an amino acid mutation at one or more active binding sites, and/or one or more sites involved in accessibility to the one or more active binding sites, optionally wherein the reference enzyme is a wild type enzyme or an enzyme with known production yield of the ketone, optionally wherein the enzyme variant is a CCD1 variant.

15.-16. (canceled)

17. A system for identifying one or more enzyme variants that produces an increased level of a ketone as compared to a reference enzyme comprising:
(a) purifying and crystalizing the reference enzyme;
(b) identifying one or more active binding sites and/or one or more sites involved in accessibility to the one or more active binding sites;
(c) introducing one or more amino acid mutations at the active binding sites and/or accessibility sites to generate the one or more enzyme variants;
(d) subjecting the one or more enzyme variants from step c) to the cell-based method of claim 1 to identify the one or more enzyme variants that produces the increased level of ketone.

18. The system according to claim 17, wherein the one or more enzyme variants identified has an increased binding affinity for a substrate and/or increased catalytic activity compared to the reference enzyme.

19. A genetically engineered enzyme variant identified using the system according to claim 17.

20. The genetically engineered enzyme variant according to claim 19, wherein the enzyme variant is an apocarotenoid pathway enzyme, optionally wherein the apocarotenoid enzyme is carotenoid cleavage dioxygenase (CCD), optionally, wherein the CCD is CCD1 or CCD4, optionally wherein the CCD is CCD1, optionally wherein the CCD1 is mutated at one or more amino acid positions, optionally wherein the CCD1 gene is a plant CCD1 gene, optionally wherein the plant CCD1 gene is selected from the group consisting of Petunia hybrida CCD1 (PhCCD1), Vitis vinifera CCD1 (VvCCD1) and Osmanthus fragrans (OfCCD1).

21.-25. (canceled)

26. The genetically engineered enzyme variant claim 20, wherein the mutation is a substitution at one or more amino acid positions selected from the group consisting of A191, A400, C354, E408, F303, F530, H241, I171, V179, L356, L423, N339, N377, Q309, Q415, R405, S189, S220, S240, T173, T242, T411, T412, T421, V227, V401, V429 and combinations thereof of a wild type OfCCD1 (SEQ ID NO: 1), or one or more amino acid positions equivalent to the positions selected from the group consisting of A191, A400, C354, E408, F303, F530, H241, I171, V179, L356, L423, N339, N377, Q309, Q415, R405, S189, S220, S240, T173, T242, T411, T412, T421, V227, V401, V429 and combinations thereof of a wild type OfCCD1 (SEQ ID NO: 1), optionally wherein the mutation at one or more amino acid positions in CCD1 is selected from the group consisting of A191G, A191 L, A191M, A400V, C354S, E408P, E408Q, F303Y, I171T, L3561, L356M and R405K and combinations thereof.

27. (canceled)

28. The genetically engineered enzyme variant according to claim 27, wherein the mutated CCD1 produces an increased β-ionone yield and decreased psi-ionone yield compared to a wild type CCD1.

29. The genetically engineered enzyme variant according to claim 19, wherein the one or more enzyme variants that produces an increased level of a ketone has an increased binding affinity for a substrate and/or increased catalytic efficiency compared to a reference enzyme.

30. The genetically engineered enzyme variant according to claim 29, wherein the substrate is β-apo-8′ carotenal.

31. The genetically engineered enzyme variant according to claim 20, wherein the CCD1 comprising the mutations at amino acid positions A191 and E408 has increased binding specificity for β-apo-8′ carotenal or increased catalytic efficiency or increased β-ionone production or combinations thereof compared to the wild type CCD1.

32. A genetically engineered CCD1 enzyme comprising one or more mutations, wherein the one or more mutations is a substitution at the one or more amino acid positions selected from the group consisting of A191, A400, C354, E408, F303, F530, H241, I171, V179, L356, L423, N339, N377, Q309, Q415, R405, S189, S220, S240, T173, T242, T411, T412, T421, V227, V401 and V429 of a wild type OfCCD1 (SEQ ID NO: 1).

33.-38. (canceled)


SEQUENCE LISTING (3 sequences)

SEQ ID NO: 1 - protein, 543 aa, Osmanthus fragrans
MGMQGEDAQRTGNIVAVKPKPSQGLTSKAIDWLEWLFVKMMHDSKQPLHYLSGNFAPVDETPPLKDLPVTGHLPECLNGEFVRVGPNPKFASIAGYHWFDGDGMIHGMRIKDGKATYVSRYVQTSRLKQEEFFGRAMFMKIGDLKGMFGLLMVNMQMLRAKLKVLDISYGIGTANTALVYHHGKLLALSEADKPYAIKVLEDGDLQTIGLLDYDKRLAHSFTAHPKVDPFTGEMFTFGYSHTPPYVTYRVISKDGAMNDPVPITVSGPIMMHDFAITENYAIFMDLPLYFKPKEMVKDKKFIFSFDATQKARFGILPRYAKNELLIKWFELPNCFIFHNANAWEEGDEVVLITCRLENPDLDMVNSTVKERLDNFKNELYEMRFNLQNGLASQKKLSVSAVDFPRVNESYTTRKQRYVYGTTLDKIAKVTGIIKFDLHAEPETGKEKLELGGNVKGIFDLGPGRFGSEAVFVPRHPGITSEEDDGYLIFFVHDENTGKSAVNVIDAKTMSPDPVAVVELPKRVPYGFHAFFVTEDQLQEQAKV

SEQ ID NO: 2 - protein, 546 aa, Petunia hybrida
MGRKESDDGVERIEGGVVVVNPKPKKGITAKAIDLLEKVIIKLMHDSSKPLHYLSGNFAPTDETPPLNDLPIKGHLPECLNGEFVRVGPNPKFAPVAGYHWFDGDGMIHGLRIKDGKATYVSRYVRTSRLKQEEFFEGAKFMKIGDLKGLFGLFTVYMQMLRAKLKILDTSYGNGTANTALVYHHGKLLALSEADKPYALKVLEDGDLQTLGMLDYDKRLLHSFTAHPKVDPVTGEMFTFGYAHEPPYITYRVISKDGIMQDPVPITIPEAIMMHDFAITENYAIMMDLPLCFRPKEMVKNNQLAFTFDTTKKARFGVLPRYAKSEALIRWFELPNCFIFHNANAWEEGDEVVLITCRLPHPDLDMVNGEVKENLENFSNELYEMRFNMKSGAASQKKLSESSVDFPRINENYTGRKQRYVYGTTLNSIAKVTGIIKFDLHAEPETGKKQLEVGGNVQGIFDLGPGRFGSEAVFVPSQPGTECEEDDGYLIFFVHDENTGKSAVNVIDAKTMSAEPVAVVELPKRVPYGFHAFFVTEEQIQEQAKL

SEQ ID NO: 3 - protein, 542 aa, Vitis vinifera
MAEKEEQGGAGVAVVDPKPSKGFTSKAVDWLEKLIVKLMYDSSQPLHYLSGNFAPVRDETPPCKNLPVIGYLPECLNGEFVRVGPNPKFSPVAGYHWFDGDGMIHGLHIKDGKATYVSRYVRTSRLKQEEYFGGAKFMRIGDLKGLFGLLMVNMQMLRAKLKILDVSYGTGTGNTALVFHRGKLLALSEADKPYVLKVLEDGDLQTLGMLDYDKRLTHSFTAHPKVDPFTGEMFSFGYSHTPPYITYRVISKDGFMHEPVPITISDPIMMHDFAITENYAIFMDLPLYFRPKEMVKEKKLIFTFDATKKARFGVLPRYAKNELHIKWFELPNCFIFHNANAWEEEDEVVLITCRLEHPDLDLVGGDVKEKLENFGNELYEMRFNMKTGIASQRKLSASSVDFPRVNESYTGRKQRYVYGTILDSIAKVTGIIKFDLHAEPDTGKSKLEVGGNVQGIFDLGVGRFGSEAVFVPREPGITSEEDDGYLIFFVHDEKTGKSYVNVIDAKTMSPDPIAIVELPNRVPYGFHAFFVTEEQLKEQAKL